(19) United States
(12) Patent Application Publication
(10) Pub. No.: US 2005/0163729 A1
(43) Pub. Date: Jul. 28, 2005

(54) Oral compositions containing peroxide and methods for use

(76) Inventors: Lynette A. Zaidel, Cranford, NJ (US); Nora C. Lin, Basking Ridge, NJ (US); David B. Viscio, Monmouth Junction, NJ (US)

Correspondence Address: COLGATE-PALMOLIVE COMPANY, 909 RIVER ROAD, PISCATAWAY, NJ, 08855, US

(21) Appl. No.: 11/043,217

(22) Filed: Jan. 26, 2005

Related U.S. Application Data
(63) Continuation-in-part of application No. 10/065,244, filed on Sep. 27, 2002.
(60) Provisional application No. 60/539,421, filed on Jan. 26, 2004.

(51) Int. Cl. A61K 7/20

(52) U.S. Cl. 424/53

(57) ABSTRACT

A peroxide-containing dual component tooth-whitening system providing enhanced whitening efficacy, minimal gingival irritation, and tooth sensitivity is described. The system is comprised of a first component containing a peroxide compound and a peroxide-compatible abrasive and a second component containing an alkaline compound. When the components are combined and contact the surface of a tooth an enhanced whitening effect is obtained with minimal tooth sensitivity and gum irritation. A desensitizer compound, a color indicator, and a stabilizing carrier can be included in the system. In certain embodiments, the composition is substantially free of chelating agents.

### CROSS REFERENCE TO RELATED APPLICATIONS

This application claims priority to provisional U.S. Patent Application No. 60/539,421, filed Jan. 26, 2004, and to U.S. patent application Ser. No. 10/065,244, filed Sep. 27, 2002 of which this application is a continuation-in-part; and the contents of each of the above are incorporated herein by reference.

### BACKGROUND OF THE INVENTION

Tooth whitening methods currently offered in dental practices often involve the use of take-home whitening kits containing a peroxide gel or paste. In one type of kit, the peroxide product is placed in a pre-fabricated tray and worn by the patient for one to two weeks in daily application times ranging from thirty minutes to overnight. In an attempt to obtain more rapid whitening effects, products containing high concentrations up to 22% carbamide peroxide are now available. Nevertheless, such products can often produce greater tooth and gingival irritation. Thus, there remains a continuing need for new peroxide-based tooth-whitening products.

### BRIEF SUMMARY OF THE INVENTION

In various embodiments, the present invention provides a dual component tooth-whitening system. The first component includes a peroxide compound and a peroxide-compatible abrasive compound. The second component includes an alkaline compound. When the first and second components are mixed with one another the resultant composition of the tooth whitening system has a pH of about 9 or greater.

In certain embodiments, the present invention provides a tooth-whitening composition that includes a peroxide compound at a hydrogen-peroxide-equivalent concentration of not greater than about 5% by weight and a peroxide-compatible abrasive compound. The composition has a pH of about 9 or greater.

In yet other embodiments, the present invention provides methods for decreasing whitening time, increasing whitening effectiveness, and/or decreasing oral irritation in a tooth-whitening process. The methods described include contacting a tooth for an effective tooth-whitening period with a tooth-whitening composition. The compositions include a peroxide compound at a hydrogen-peroxide-equivalent concentration of not greater than about 5% by weight and a peroxide-compatible abrasive compound. The composition has a pH of at least about 9.

In another embodiment, the present invention provides a dual component tooth-whitening system. The system includes a first component having a peroxide compound and a peroxide-compatible abrasive compound. The system also includes a second component having an alkaline compound and one or more stabilizing compounds compatible with the alkaline compound. Upon combination of the first and second components, a resultant tooth-whitening composition has a pH of about 9 or greater.

The compositions and methods of this invention afford advantages over prior art whitening compositions by providing effective tooth-whitening in a decreased time by using a tooth-whitening composition. Further, the tooth-whitening systems and methods of the present invention provide effective tooth-whitening in a decreased time while minimizing gingival irritation. Additional uses, benefits, and embodiments of the present invention are apparent from the description set forth herein.

### DETAILED DESCRIPTION OF THE INVENTION

All compositional percentages and amounts in the specification and claims are by weight unless otherwise indicated. Reference herein to amounts by weight can also be expressed as (w/w) which is intended to mean the ratio of the number of grams of a particular component of a composition to the total number of grams of that composition.

As used herein, the term “about,” when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value, except where otherwise indicated. As referred to herein, the word “substantially,” when applied to a characteristic of a composition or method of this invention, indicates that there may be variation in the characteristic without having significant effect on the chemical or physical attributes of the composition or method.

The present invention, in various embodiments, can involve methods and compositions for enhancing tooth-whitening with compositions containing one or more peroxide compounds and one or more abrasive compounds, wherein the compositions can have a pH of at least about 9. Such compositions are stable during storage, and provide an accelerated tooth-whitening process, which increases the whitening activity of the peroxide compounds and decreases oral irritation. Reference herein to “a tooth” is intended to include both the singular (tooth) and plural (teeth) of a mammalian subject, which includes humans and other warm blooded higher level vertebrate animals, such as felines and canines. The tooth-whitening effect of the compositions is detectable by visual observation or by measurement using any of various instruments following a given application period or following successive application periods of the same duration.

Tooth whiteness can be visually observed or measured by any of various instruments. One such instrument can be a calorimeter such as, for example, a Minolta portable Chromameter such as model CR-400 (Minolta Corp. Ramsey, N.J., Unites States). This calorimeter can be programmed to measure Hunter lab values of “L”, “a,” and “b” in which “L” values represent lightness and “a” and “b” values represent the chromaticity coordinates. Lightness “L” values can represent dark to light color in which a value of 0 represents black and a value of 100 represents white. Green to red can be expressed by the “a” value, the more positive value representing more red, and the more negative value representing more green color (−80 represents green and 100 represents red). Blue to yellow can be expressed by the “b” value, a more positive value representing more yellow color in the sample (−80 represents blue and 70 represents yellow). Typically “L” values are used to measure tooth-whiteness.

In certain embodiments, the invention provides methods of enhanced tooth-whitening with a composition comprising a peroxide compound and one or more abrasive compounds, the composition having a pH of about 9 or greater. Any one or more of an increased rate of tooth-whitening, increased effectiveness of tooth-whitening and decreased oral irritation are produced in comparison to that produced by a tooth-whitening composition containing the same or greater concentration of the peroxide compound at a pH of about 7 or less in the absence of the one or more abrasive compounds.

In various embodiments, where the tooth-whitening composition is provided in a dual component system, a first component comprises the peroxide compound in an aqueous vehicle at a pH of about 7 or less, and a secon separately maintained component comprises a stable alkaline compound in a stable carrier that preferably has a pH of about 7 or greater. Thus, upon mixing the first and second components, it is preferred that the resulting tooth-whitening composition has a pH of about 9 or greater. It is also possible to initially prepare the composition containing both the peroxide compound and the alkaline compound such that the composition has a pH of about 9 or greater.

In various embodiments, the present invention provides an improved tooth-whitening oral composition. Preferably, the present invention includes a dual component oral composition comprising both a first and a second component which are separately maintained. The first component preferably comprises a peroxide compound, a peroxide-compatible abrasive compound, and an orally acceptable carrier, compatible with the peroxide compound. The second component preferably comprises an alkaline compound and an orally acceptable carrier compatible with the alkaline compound.

As used herein, an “orally acceptable carrier” refers to a material or combination of materials that are safe for use in the compositions of the present invention, commensurate with a reasonable benefit/risk ratio, with which the peroxide compound and/or alkaline compound may be associated. Preferably, the carrier does not substantially reduce the efficacy of the peroxide compound, alkaline compound, or the other active materials of the present compositions. An orally acceptable vehicle carrier may be any carrier toxicologically suitable for use in the oral cavity. Such orally acceptable carriers include the usual components of toothpastes, tooth powders, prophylaxis pastes, rinses, gels and the like, and are more fully described hereinafter. Selection of specific carrier components is dependant on the desired product form and by way of example may include whitening compositions (such as those used in conjunction with professional whitening kits), dentifrices, gels, paint-on compositions, and the like.

Thus, the first and second components (and/or the composition as a whole) can be, independently, in any form, such as, e.g., a liquid, a gel, a suspension, a paste, a cream, a semi-solid, a solid, or a form that changes with variations in chemical or physical environment, e.g., mixture with another compound and/or change of temperature (room temperature to body temperature).

In various embodiments the dual component oral composition of the invention includes both a first and a second component that separately maintained. Maintaining the components separately requires only that the components are maintained in such a way as to substantially prevent the interaction of the alkaline compound and the peroxide compound. This can be accomplished through any means known or to be discovered in the art and includes chemical, physical, and mechanical means of separation of any combination of these. For example, the first and second components may be combined but the alkaline compounds and/or the peroxide compound are separately maintained by wrapping or encapsulating one or both in a film, coating, capsule, micelle, etc.

In various embodiments, it is contemplated that the first and second components are combined to form a mixture that produces an oral composition having a pH of about 9 or greater prior to and/or as it is applied to the tooth surface. By “combining” the first and second components, it is meant that the first and second components are substantially homogenously mixed or that predetermined portions of the first and second components are intermingled and mixed. For example, the first and second components can be combined in a 1:1 ratio by either weight or volume. In certain embodiments, the pH of the first component may be in a range of about 4 to about 7, and the pH of the second component may be in a range of at least about 9 to about 13.

It should be noted that in certain embodiments, the physical separation of the peroxide compound and the alkaline compound may occur by chemical or physical means (e.g., encapsulation of one or both compounds, where the encapsulation dissolves within the oral cavity) and the respective compounds can be incorporated into a single tooth-whitening component capable of both stable storage and delivery to the tooth surface at a pH of greater than 9.

Typically, equal amounts of the first component and the second component are combined in such a manner to effect mixing. Such mixing results in an increase in the pH of the mixture to about 9 or greater, about 9.5 or greater, about 10 or greater, about 11 or greater, or about 12 or greater up to a pH of about 13 or greater. In certain embodiments, it is preferred that the pH is about 9 to about 11. In various aspects of the embodiments of the present invention involving a dual component system, the first and second components can be combined for not more than about fifteen minutes, not more than about ten minutes, not more than about five minutes, not more than about two minutes, or just prior to, contacting a tooth with the composition.

Thus, the enhanced whitening effect of the compositions of the invention can be achieved at a reduced concentration of peroxide compounds when compared to concentrations conventionally used. The concentration of the peroxide compound in the composition of the invention is a reduced concentration compared to a comparative composition, i.e., the methods in embodiments of the invention use a reduced concentration of a peroxide compound(s) to achieve similar whitening effects during the course of a whitening period, having a similar effectiveness of whitening and a greater degree of absence of oral irritation.

Concentrations of peroxide compounds conventionally used in prior art compositions may be, for example, 7% hydrogen peroxide by weight or greater, 7.5% hydrogen peroxide by weight or greater, or an amount of a peroxide compound sufficient to produce a hydrogen peroxide ion or an organic peroxide ion in an amount equivalent to the hydrogen peroxide ion delivered by 7% by weight hydrogen peroxice or 7.5% by weight hydgrogen peroxide under the same conditions. Such equivalence is referred to herein as a “hydrogen-peroxide-equivalent concentration” for a given peroxide compound and can be measured using routine analytical techniques.

For example, carbamide peroxide at a concentration of 10% by weight in an aqueous solution delivers an amount of hydrogen peroxide ion equivalent to an aqueous solution of hydrogen peroxide at a concentration of about 3% by weight. Thus, for carbamide peroxide, a hydrogen-peroxide-equivalent concentration of 3.5% by weight is equivalent to a carbamide peroxide concentration of about 12% by weight, a hydrogen-peroxide-equivalent concentration of 5% by weight is equivalent to a carbamide peroxide concentration of about 17% by weight and a hydrogen-peroxide-equivalent concentration of 7% by weight is equivalent I to a carbamide peroxide concentration of about 22% by weight.

In the present invention, the peroxide compounds may be present in any amount sufficient to produce a whitening effect. Preferably, the peroxide compounds are present in an amount that is a hydrogen-peroxide-equivalent concentration of not more than about 3% by weight; not more than about 3.5% by weight; not more than about 4% by weight; not more than about 4.5% by weight; not more than about 5% by weight; not more than about 5.5% by weight; not more than about 6% by weight; not more than about 6.5% by weight; not more than about 7.0% by weight; not more than about 7.5% by weight; and not more than about 8.0% by weight.

The peroxide compound(s) selected for use in the invention may be any of a variety of peroxide-based whitening agents that deliver a hydrogen peroxide ion or an organic peroxide ion. Such compounds include, for example, hydrogen peroxide, organic peroxide compounds, hydrogen peroxide generating compounds, organic peroxide generating compounds and combinations thereof.

Organic peroxide compounds include, for example, urea hydrogen peroxide (carbamide peroxide), glyceryl hydrogen peroxide as well as groups of peroxides classified according to the number and kind of organic functional groups attached to the oxygen atoms, such as, for example, alkyl hydrogen peroxide (R—O—O—H), dialkyl hydrogen peroxide (R—O—O—R′), peroxy acids (RCO—O—O—H), peroxy esters (RCO—OOR′), and diacyl peroxides (R—CO—O—O—CO—R′).Among such peroxides used in dental whitening are the diacyl peroxide, benzoyl peroxide and the peroxy acid monoperoxyphthalate.

A hydrogen peroxide generating compound suitable for use in the invention may be for example, alkali metal and alkaline-earth metal persulfate, dipersulfate, percarbonate, perphosphate, perborate, and persilicate salts such as, for example, sodium persulfate, sodium dipersulfate, sodium percarbonate, sodium perphosphate, sodium perborate, sodium persilicate, potassium persulfate, potassium dipersulfate, potassium percarbonate, potassium perphosphate, potassium perborate, potassium persilicate, calcium persulfate, calcium dipersulfate, calcium percarbonate, calcium perphosphate, calcium perborate, calcium persilicate salts as well as sodium peroxide, potassium peroxide and calcium peroxide and combinations of all of the above hydrogen peroxide generating compounds.

The second component of the invention includes an alkaline compound. The alkaline compound can be, for example, an alkali metal, ammonium or alkaline earth metal compound such as, for example, sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate or combinations thereof; an organic amine such as urea, alkanolamines such as monoethanolamine, diethanolamine, triethanolamine, mono(iso)propanolamine, di(iso)propanolamine, tri(iso)propanolamine or 2-amino-2-methylpropanol; alkanediolamines such as 2-amino-2-methyl-1,3-propanediol or 2-amino-2-ethyl-1,3-propanediol; alkanepolyamines such as tris(hydroxymethyl)aminomethane or N,N,N′N′-tetrakis(2-hydroxypropyl)ethylenediamine; alkylamines such as di(2-ethylhexyl)amine, triamylamine or dodecylamine; amino ethers such as morpholine; and mixtures thereof.

The alkaline compound may be present in the second component of the tooth-whitening composition at a concentration of about 0.1% by weight to about 30% by weight, about 0.2% by weight to about 10% by weight, about 0.5% by weight to about 2% by weight or about 0.5% by weight to about 0.75% by weight.

In various embodiments, one or more abrasive compounds can be included in the tooth-whitening compositions of the present invention. One or more of a selected abrasive compound(s) can be present in either or both of the first component or the second component. In various embodiments, the abrasives are present in the overall composition at a concentration of greater than about 20%. Any abrasive suitable for delivery to the oral cavity known to one of skill in the art may be used. Abrasives may include silica compounds and alumina compounds. Such silica compounds include for example, hydrated silica, such as SORBOSIL™ AC-35, marketed by INEOS Silicas Ltd (Warrington, United Kingdom; formerly Crosfield Chemicals), or ZEODENT® 115 from Huber Company (Edison, N.J., United States). Alumina compounds can include, for example, alumina trihyhydrate, aluminum silicate, calcined alumina and mixtures thereof. Other abrasives include hydroxyapatite, sodium metaphosphate, potassium metaphosphate, tricalcium phosphate, calcium carbonate, sodium bicarbonate, bentonite, dicalcium phosphate dihydrate, anhydrous dicalcium phosphate, calcium pyrophosphate and mixtures thereof.

In various embodiments of the present invention, the second component of the dual phase tooth whitening compositions of the present invention optionally comprises one or more of an abrasive. However, in various preferred embodiments, the first component preferably includes a abrasive that is chemically inert with respect to the peroxide compound (a “peroxide-compatible abrasive”), i.e., one that does not adversely react with the peroxide compound to cause substantial decomposition of the peroxide compounds. Examples of suitable peroxide-compatible abrasives include calcium phosphate abrasive compounds (see for example, U.S. Pat. No. 5,171,564 to Nathoo, et al., the contents of which are incorporated herein by reference). Other examples of such peroxide compatible abrasives include dicalcium phosphate dihydrate, anhydrous dicalcium phosphate, calcium pyrophosphate and mixtures thereof. The peroxide-compatible abrasive can be present in the first component of the tooth-whitening composition at a concentration of at least about 10% by weight or at a concentration of at least about 20% by weight. In certain embodiments, the first component optionally comprises an abrasive selected from the broader class of abrasives listed above (including those that are not “peroxide-compatible”) at a relatively low concentration that does not significantly react with or detract from the efficacy of the peroxide compound.

As previously discussed above, the first and the second component of the present invention can be provided in any conventional orally acceptable carrier and can independently be in a liquid, semi-solid, or solid phase. As recognized by one of skill in the art, the compositions of the present invention optionally include other materials in addition to those components previously described, including for example, viscosity modifiers, diluents, surface active agents, such as surfactants, emulsifiers, and foam modulators, additional pH modifying agents, humectants, thickeners, emollients, and moisturizers, mouth feel agents, sweetening agents, flavor agents, colorants, preservatives and combinations thereof. It is understood that while general attributes of each of the above categories of materials may differ; there may be some common attributes and any given material may serve multiple purposes within two or more of such categories of materials. Preferably, such carrier materials are selected for compatibility and stability with both the peroxide compound and peroxide-compatible abrasive, if included in the first component, or with the alkaline compound, if included in the second component.

The dual component system can be in the form of a kit which includes the first and second components along with instructions for combining the first and second components and/or instructions as to the method of use of the first and second components and the mixture thereof. Such instructions as to the method of use can include the amount to be used, time period for applying the compositions, schedule for repeated application and the like.

The whitening composition of the present invention may be provided in a multi-component dentifrice that is packaged in a suitable dispensing container in which the first and second components are maintained separately and from which the separated components may be dispensed synchronously as a combined ribbon for application to a toothbrush. Under such circumstances, it may be preferable that each component is formulated to have similar rheological characteristics, so that the two components may be simultaneously co-extruded in the desired predetermined amounts when separately housed in a multi-compartmented tube or pump device. Such containers are well known in the art. An example of such a container is a two compartment dispensing container, such as a pump or a tube, having collapsible sidewalls, as disclosed in U.S. Pat. No. 6,447,756 to Dixit, et al.; U.S. Pat. No. 4,487,757 to Kiozpeoplou; and U.S. Pat. No. 4,687,663 to Schaeffer (the contents of each of which are incorporated herein by reference); where, the tube body is formed from a collapsible plastic web such as polyethylene or polypropylene and is provided with a partition within the container body defining separate compartments in which the physically separated components are stored and from which they are dispensed through a suitable dispensing outlet.

The compositions of the present invention, including the tooth-whitening compositions and the first and second components of two-component systems, may contain a thickener system containing one or more thickening agents which imparts a high viscosity to the composition. In various embodiments, the oral composition preferably has a typical viscosity of from less than about 200 centipoise (cP) up to about 10,000 cP or greater or about 1000 cP up to about 9000 cP or about 4000 cP to about 6000 cP.

In various embodiments, such as for toothpastes, creams and gels, the oral composition contains a natural or synthetic thickener or gelling agent, which other than silica thickeners, include natural and synthetic gums and colloids. Any thickener known or to be developed in the art may be used: however, suitable thickeners include naturally occurring polymers (such as carrageenan or xanthan gum), synthetic thickeners such as polyglycols of varying molecular weights, and cellulose polymers (such as hydroxyethyl cellulose or hydroxypropyl cellulose). Other thickeners include natural and synthetic clays such as hectorite clays, lithium magnesium silicate (laponite) and magnesium aluminum silicate (Veegum). Other suitable thickeners are synthetic hectorite, a synthetic colloidal magnesium alkali metal silicate complex clay available, for example, as LAPONITE® (e.g., CP, SP 2002, D) marketed by Laporte Industries Limited.

Thus, a non-limiting list of suitable thickeners includes Irish moss, gum tragacanth, xanthan gum, gum arabic, dextran, agar, furcellan algin, chitin, chitosan, carrageenan, starch, polyvinylpyrrolidone, hydroxyethyl propyl cellulose, hydroxybutyl methyl cellulose, hydroxypropyl methyl cellulose, hydroxyethyl cellulose (e.g., available as NATROSOLO®), sodium carboxymethyl cellulose, and colloidal silica such as finely ground SYLOID® (e.g., 244). In certain embodiments, the thickening agent is present in the oral composition in amounts of about 0.1 to about 20%, preferably about 0.5 to about 15%.

The thickening agent for use in the composition can include a hydrophilic block copolymer of polyethylene oxide and polypropylene oxide such as a PLURONIC® compound, for example PLURONIC® F127 which is a trade name of BASF obtained therefrom (BASF Corporation, Mount Olive, N.J., United States). P LURONIC® F127 has a molecular weight of about 4000. This substance can be present in the compositions at a concentration of about 5% by weight about 10% by weight, about 15% by weight, about 20% by weight, about 25% by weight, about 30% by weight or greater. In certain embodiments, the PLURONIC® F127 concentration in the first component containing the peroxide compound can be about 20%.

Polyethylene oxide polymers of high molecular weight can also be used as a thickener. Such polyethylene oxide thickeners have a number average molecular weight of about 50,000 to about 5,000,000. One or more high molecular weight polyethylene oxide polymers can be present in a concentration range of about 0% to about 30% by weight, about 10% by weight to about 25% by weight or about 15% by weight to about 20% by weight.

One or more of glycerin, sorbitol, and low molecular weight polyethylene glycol(s) can also be included as thickening agents in the compositions as carrier materials which can also impart effects on viscosity. The polyethylene glycol is a nonionic polymer of ethylene oxide. The polyethylene glycol when present in the compositions of the present invention can have a number average molecular weight of about 200 to about 1000, about 400 to about 800 or about 600. The glycerin, sorbitol and polyethylene glycol can each independently be present in amounts of about 0% to about 30% by weight, about 5% by weight to about 25% by weight, about 10% by weight to about 20% by weight, or about 15% by weight to about 20% by weight. In one embodiment, glycerin and polyethylene glycol (600) are present in amounts of about 5% by weight and about 15% by weight, respectively, in the first component composition, in amounts of about 5% by weight and about 10% by weight, respectively, in the second component composition and in amounts of about 5% by weight and about 12.5% by weight, respectively, in the tooth-whitening composition.

In an embodiment, the composition of the invention may be prepared by combining a first component that has a relatively high peroxide content (such as, e.g., a concentration of about 5% by weight to about 8% by weight of a hydrogen peroxide equivalent) and a corresponding relatively high concentration of the alkaline compound(s) such that the resultant composition has a pH of, e.g., about 9 or greater. In these circumstances, it may be desirable to include within the second component a compound or mixture of compounds that aids or enhances the long-term chemical stability of the alkaline compounds. Any such stabilizing compounds or agents may be used. Advantageously, such compounds may have the added advantage of being dually functional, i.e., it may increase long-term chemical stability of the alkaline compound while simultaneously contributing another property of the component or overall compositions (for example, an agent that alters rheological properties, a flavorant, a thickener, an emulsifier, etc.)

It may be desirable to include within the second component any one or more naturally occurring polymers, such as marine colloids like agar, furcellan algin, chitin, chitosan, or carrageenan; plant and microbial gums, such as xanthan gum, gum arabic, dextran, Irish moss, or gum tragacanth; starch, polyglycols of varying molecular weights, and cellulose polymers (such as hydroxyethyl cellulose or hydroxypropyl cellulose), polyvinylpyrrolidone, hydroxyethyl propyl cellulose, hydroxybutyl methyl cellulose, hydroxypropyl methyl cellulose, hydroxyethyl cellulose (e.g., available as NATROSOL®), sodium carboxymethyl cellulose, an colloidal silica such as finely ground SYLOID® (e.g., 244).

These compounds may be present in any amount, as needed to stabilize and/or impart other desired effects in the second component or the composition as a whole. It may be preferable to include two of the selected compounds in a range of a first stabilizing compound to a second stabilizing compound of between about 2:1 to about 1:4, preferably between about 1:1 to about 1:2, more preferably at about 1:1.5.

For example, in one preferred embodiment the second component includes a first stabilizing compound of sodium carboxymethyl cellulose (CMC) and a second stabilizing compound of xanthan gum. Sodium CMC is present in an amount of about 0.25 and 1%, preferably about 0.5% in the second component, and xanthan is present in an amount of about 0.5% and 1%, preferably about 0.7% in the second component. Thus, a ratio of CMC to xanthan ranges from about 2:1 to 1:4. In an embodiment, the second component includes CMC as a first stabilizing compound at about 0.5% by weight and xanthan as a second stabilizing compound at about 0.7%, corresponding to a ratio of approximately 1:1.4.

In various embodiments, the overall composition of the present invention contains added water in an amount of about 5% by weight to about 40% by weight, about 10% by weight to about 30% by weight or about 5% by weight to about 20% by weight.

Surfactants or surface active agents can also be included in each or both of the first or second components of the present invention as solubilizing, dispersing, and/or emulsifying agents. Such surfactants can include nonionic surfactants such as, for example, Tween 20 or anionic surfactants such as, for example, sodium lauryl sulfate or sodium dodecyl sulfate.

Each or both of the first or second components o f the present invention may contain flavoring substances at a concentration of about 0.05% by weight to about 5% by weight. Such flavoring substances include, by way of example, an essential oil, extract or flavoring aldehyde, ester or alcohol that imparts a flavor of spearmint, peppermint, wintergreen, sassafras, clove, sage, eucalyptus, marjoram, cinnamon, lemon, lime, grapefruit, orange, apple, pear, peach, strawberry, vanilla, miswak, cherry, cola, apricot, watermelon, banana, coffee, cocoa, menthol, carvone, anethole or combinations thereof.

One or more sweetening agents can also be included in each or both of the first or second components of the present invention. Such sweetening agents can include natural or synthetic sweeteners, sodium saccharin, sodium cyclamate, xylitol, aspartame, and the like. It may be desirable to include the sweeteners in a concentration of about 0.01% by weight to about 1% by weight of either of the first or second components.

The compositions of the present invention can also be formulated to contain one or more desensitizing agents to reduce tissue sensitivity and irritation upon application of the tooth-whitening compositions. Such desensitizers can be included in the first or second component, or both. Suitable desensitizers include, for example, potassium salts of weak acids, such as potassium citrate, potassium chloride, potassium tartrate, potassium bicarbonate, potassium oxalate, potassium nitrate as well as strontium salts and eugenol (4-allyl-2-methoxyphenol).

One or more of the desensitizers can be present in tooth-whitening composition of the present invention. For example, desensitizers can be present in the overall composition at a concentration of about 0.05% by weight to about 0.1% by weight or about 0.1% by weight to about 0.25% by weight of eugenol and about 1% by weight to about 10% by weight or about 3% by weight to about 6% by weight for potassium salts of weak acids such as, for example potassium nitrate.

One or more redox color indicators that are oxidized by hydrogen peroxide can also be included in the tooth-whitening composition. The indicators may change colors upon contact with the peroxide compound, and can be used to signal the user when the effective whitening period is completed. Such a color indicator can be included in the second component of the dual-component system. In certain embodiments, the color indicator may be included in the first component where it is physically isolated from with the peroxide compound until use (e.g., where the color indicator is encapsulated in a protective coating that dissolves upon use in the oral cavity).

The color indicator may be a food color additive certified under the Food Drug & Cosmetic Act for use in food and ingested drugs, including dyes such as FD&C Red No. 3 (sodium salt of tetraiodofluorescein), FD&C Red No. 40 (disodium 6-hydroxy-5-((2-methoxy-5-methyl-4-sulfophenyl)azo)-2-naphthalene- sulfonate), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-Δ-3,5-cyclohexadienimine], FD&C Blue No. 1 (disodium salt of dibenzyldiethyldiaminotriphenylcarbinol trisulfonic acid of indigotin).

The color indicator(s) may preferably be present at a concentration of about 0.005% by weight to about 0.5% by weight or about 0.025% by weight to about 0.15% by weight of the component.

Agents that chelate metal ions can also be present in the compositions of the present invention. Such chelating agents include sodium acid pyrophosphate, sodium phosphate, disodium calcium ethylenediamine tetraacetic acid, phosphoric acid, citric acid, sodium citrate, potassium citrate, sodium pyrophosphate, potassium pyrophosphate, disodium ethylenediamine tetraacetic acid and the like. The chelating agents can be incorporated into the compositions of the present invention in an amount of about 0.1% by weight to about 8% by weight or about 0.5% by weight to about 3% by weight. In certain embodiments, the tooth-whitening compositions of the present invention are substantially free of chelating agents to decrease potential sensitivity in the oral cavity.

Anti-foaming agents such as simethicone can also be present in either or both of the components of the present invention. Suitable concentrations may be about 0% by weight to about 0.1% by weight of the overall tooth-whitening composition.

In certain embodiments, the tooth-whitening compositions of the present invention may include an additional active agent other than the peroxide compound and the densensitizing compound. If added, the additional active agent should not substantially react with or substantially detract from the whitening efficacy of the peroxide compound or any other ingredients of the composition.

For example, if desired one may include in the tooth-whitening composition of the invention an “oral active agent” that is operable for the prevention or treatment of a condition or disorder of hard or soft tissue of the oral cavity, the prevention or treatment of a physiological disorder or condition, or to provide a cosmetic benefit (e.g., to the teeth, gingiva or other hard or soft tissue of the oral cavity). Alternatively, or in addition, the active agent may be a “systemic active agent” that is operable to treat or prevent a disorder which, in whole or in part, is not a disorder of the oral cavity. Thus, compositions of the present invention may also be used for the treatment or prevention of systemic disorders, such as the improvement of overall systemic health characterized by a reduction in risk of development of systemic diseases, such as cardiovascular disease, stroke, diabetes, severe respiratory infection, premature and low birth weight infants (including associated post-partum dysfunction in neural/developmental function), and associated increased risk of mortality. Such methods include those disclosed in U.S. patent Publication 2003/0206874, Doyle et al., published Nov. 6, 2003, the contents of each of which are incorporated herein by reference.

Suitable oral care actives agents include anticaries agents, tartar control agents, periodontal actives, breath freshening agents, malodour control agents, nutrients, anti-inflammatory agents, salivary stimulants, anti-adhesion agents, plaque dispersing agents, and combinations thereof. It is understood that while general attributes of each of the above categories of actives may differ, there may be some common attributes and any given material may serve multiple purposes within two or more of such categories of actives. Active agents among those useful in the methods and compositions of the invention are disclosed in the above referenced U.S. patent Publication No. 2003/0206874, Doyle et al., published Nov. 6, 2003; as well as in U.S. Pat. No. 6,290,933, Durga et al., issued Sep. 18, 2001; and U.S. Pat. No. 6,685,921, Lawlor, issued Feb. 3, 2004. Active agents useful herein are optionally present in the compositions of the present invention in safe and effective amounts.

Certain embodiments of the tooth-whitening composition of the present invention may include fluoride compounds having anti-caries activity. Such fluoride compounds include fluoride salts such as, for example, sodium fluoride, potassium fluoride, cuprous fluoride, stannous fluoride, stannous chlorofluoride, sodium fluorosilicate, ammonium fluorosilicate, sodium monofluorophosphate, alumina mono-fluorophosphate and alumina difluorophosphate. The fluoride compounds, if present, can be at a concentration sufficient to release fluoride ion in an amount of about 15 to about 1500 ppm.

In various embodiments, the present invention can provide methods for whitening a tooth in a mammal. The present invention, in various embodiments, can also include methods for decreasing whitening time, increasing whitening effectiveness or decreasing oral irritation in a tooth-whitening process. The methods of the present invention generally comprise contacting a surface of a tooth for an effective tooth-whitening period with a tooth-whitening composition. An “effective tooth-whitening period” represents the time period where the tooth-whitening compositions are applied to the tooth surface to achieve a detectable whitening effect as measured by any method known to one of skill in the art, including by visual observation or by colorimeter analysis, as described previously above.

An effective tooth-whitening period includes both continuous application of the tooth-whitening composition to the tooth surface, as well as discontinuous application to the tooth surface. An effective tooth-whitening period ranges from thirty seconds to one-thousand minutes or more. An effective tooth-whitening period encompasses both single applications for a longer duration (e.g., a one-time application for an hour), as well as repeated, intermittent applications for shorter periods (e.g., multiple applications for fifteen minute intervals). Such repeated applications can be performed one or more times during the day such as, for example, once a day, twice a day or three times a day or less frequently such as, for example once every two days, once every three days or once a week. The application period can continue, for example, for about one week, about two weeks, about three weeks, or about four weeks or longer.

The methods can comprise contacting a tooth for an effective tooth-whitening period with a tooth-whitening composition comprising a peroxide compound and a peroxide-compatible abrasive compound. The pH of the tooth-whitening composition can be at least about 9 and the peroxide compound can be present at a hydrogen-peroxide-equivalent concentration of not greater than about 7.5%, not greater than about 5% by weight, or not greater than about 3.5% by weight. The tooth-whitening composition produces a decrease in whitening time, an increase in whitening effectiveness or a decrease in oral irritation or any combination thereof in comparison to a comparative composition having a peroxide compound concentration of about 7% by weight or greater and a pH of about 7 or less.

In various aspects of this embodiment, the effective tooth-whitening period associated with methods of the present invention is less than that a comparative tooth whitening period required for a comparative composition, which may comprise hydrogen peroxide at a concentration of about 7% by weight or greater and a pH of about 7 or less, for example.

In various aspects of this embodiment, the tooth-whitening composition can be prepared from a dual component system comprising first and second components in which the first component may include a peroxide compound and a peroxide-compatible abrasive compound and the second component may include an alkaline compound.

The invention can be further understood by reference to the examples which follow.

### EXAMPLE 1

This Example illustrates a two component tooth-whitening system in which the concentration of the peroxide compound in the tooth-whitening composition formed by combining the two components is about 3.5% by weight.

The composition of the first component and the second component are as shown in Table 1.

TABLE 1
Tooth-Whitening Composition having 3.5% H₂O₂

| | Component | |
|---|---|---|
| | A | B |
| | Peroxide Component | Alkaline Component |
| Ingredients | Weight % | Weight % |
| Deionized Water | 14.3 | 31 |
| Hydrogen peroxide | 20 | — |
| (35% solution) | | |
| Potassium Nitrate | — | 6 |
| Glycerin | 5 | 5 |
| Polyethylene glycol 600 | 15 | 10 |
| PLURONIC ® F-127 | 20 | 17 |
| Saccharin | 0.2 | — |
| Sodium hydroxide | — | 0.5-0.75 |
| FD&C Yellow #6 | — | 0.025-0.15 |
| Eugenol | 0.25 | — |
| Calcium pyrophosphate | 25 | 30 |
| Sodium lauryl sulfate | — | 0.4 |
| Flavor | 0.25 | — |

Components A and B were prepared in a Ross mixer (Charles Ross & Son Company, Hauppauge, N.Y., United States) as follows:

Component A: Water, saccharin and 30% of the total volume of 35% hydrogen peroxide were stirred in a beaker until the saccharin dissolved. PEG 600 and glycerin were added, and the mixture was placed in the Ross mixer along with PLURONIC® F127 and stirred at high speed under vacuum until a clear gel formed (approximately forty-five minutes). Calcium pyrophosphate was added and the mixture stirred at medium speed for another ten minutes under vacuum. The remaining 70% of the peroxide volume was added and stirred ten minutes at medium speed. Next, the flavor was added, and the mixture was stirred five minutes at low speed. An extrudable paste having a pH of 6.0 was obtained.

Component B: Water, sodium hydroxide, potassium nitrate, and FD&C Yellow No. 6 dye were mixed in a beaker until the potassium nitrate dissolved. PEG 600 and glycerin were added to the aqueous phase, and the mixture was placed in the Ross mixing pot along with PLURONIC® F127. The mixture was stirred at high speed under vacuum until a clear gel formed (approximately forty-five minutes). Calcium pyrophosphate, sodium lauryl sulfate, sodium bicarbonate, and sodium hydroxide were added and the mixture stirred at medium speed for another ten minutes under vacuum. An extrudable paste having a pH of 9.6 was obtained.

The two components were maintained in separate vessels.

### EXAMPLE 2

This example illustrates the in vitro whitening effect of the tooth-whitening composition of Example 1 containing peroxide compound at a concentration of 3.5% by weight and calcium pyrophosphate at a concentration of 27.5%.

Three compositions were tested. The composition was prepared by mixing component A with component B of Example 1 in approximately equal proportions.

Comparative composition (designated “C”) was a simple pluronic gel prepared at approximately the same pH and peroxide concentration as the composition of Example 1, but having water in place of the calcium pyrophosphate. Comparative composition (designated “C1”) was a commercially available whitening composition comprised of a thickened Pluronic gel containing about 3.5% hydrogen peroxide at a pH of 5.8 in addition to calcium pyrophosphate and dicalcium phosphate.

Extracted, naturally-stained human teeth were pumiced and initial color determined using a Minolta Chromameter CR-241. Three teeth each were immersed for fifteen minutes at 37° C. in each of the three tooth-whitening compositions. These fifteen minute treatments were repeated eight times for a total contact time of two hours. Tooth shade was measured midway and at the end of the total treatment period. The increase in tooth whiteness, represented as ΔE values, was calculated using the following formula:
ΔE=[ΔL]²+(Δa)²+(Δb)²]^{1/2}

wherein “L”, “a” and “b” are Hunter lab values in which “L” values represent lightness and “a” and “b” values represent the chromaticity coordinates as described above. Higher values of ΔE, indicate higher levels of tooth whiteness achieved. Results are shown in Table 2 below.

TABLE 2
In vitro Immersion Study

ΔE

| Composition | pH | 4 treatments | 8 treatments |
|---|---|---|---|
| Example 1 | 9.8 | 3.48 ± 1.28 | 5.21 ± 1.52 |
| Comparative | 9.8 | 1.61 ± 0.50 | 2.17 ± 0.56 |
| Example C | | | |
| Comparative | 5.8 | 2.60 ± 0.47 | 4.23 ± 0.97 |
| Example C1 | | | |

As shown in the table, the tooth-whitening effect of the composition of Example 1 was greater than that of comparative composition C at both measurement points. The comparative composition C contained 3.5% by weight hydrogen peroxide as did the composition of Example 1; however, the composition of Example 1 also contained calcium pyrophosphate at a concentration 27.5%. This suggests that the calcium pyrophosphate contributed to the tooth-whitening effect of the composition of Example 1. Since the majority of the extrinsic staining was removed from the sample teeth with pumice, the improved whitening produced by calcium pyrophosphate is believed to result from removal of intrinsic tooth stain. Thus, the data above show that the calcium pyrophosphate provided a beneficial whitening effect in addition to its effect on surface polishing and extrinsic stain removal.

The whitening effect produced by the composition of Example 1 at pH 9.8 was greater than that produced by comparative composition, C1, which contained the same level of hydrogen peroxide at a concentration of 3.5% and similar level of calcium pyrophosphate but at a lower pH (pH 6), thus illustrating the favorable effect of increased pH on whitening efficacy.

### EXAMPLE 3

This example illustrates the in vivo testing of the tooth-whitening composition of Example 1 containing peroxide compound at a concentration of 3.5% by weight and calcium pyrophosphate at a concentration of 27.5%.

The whitening efficacy of the composition of Example 1 was also compared to comparative composition C2 in a two-week clinical study. Comparative composition “C2” is a commercially available whitening composition comprising a thickened Pluronic gel which contains about 7.5% hydrogen peroxide at a pH of about 9 and no added abrasive compounds. One cell used comparative composition C2 thirty minutes twice daily and the other cell used the composition of Example 1 for twenty minutes once daily. Tooth shade was evaluated after fourteen days using a Vita shade guide. The results are summarized in the Table 3.

TABLE 3
Clinical Testing of Composition of Example 1

| | | | | |
|---|---|---|---|---|
| | Hydrogen | | | |
| | Peroxide | No. of | Total Wear | |
| Composition | Concentration | Subjects | Time | Shade Change |

| | | | | |
|---|---|---|---|---|
| Example 1 | 3.5% | 13 | 4.7 hrs. | 4.85 ± 2.48 |
| Comparative | 7.5% | 10 | 14.0 hrs. | 4.56 ± 2.65 |
| Example C2 | | | | |

As shown in the Table, there was no significant difference between tooth shade changes. Thus the composition of Example 1 provides the same whitening effect as comparative example C2, but at half the peroxide level and ⅓ the total wear time. In addition, subjects using comparative composition C2 reported significantly (p=0.038) more gum irritation than those using the composition of Example 1.

### EXAMPLE 4

This Example illustrates a two component tooth-whitening system in which the concentration of the peroxide compound in the tooth-whitening composition formed by combining the two components is about 5% by weight, and the alkaline component (Component B) has a stabilizing compound and silica abrasives.

The composition of the first component and the second component are as shown in Table 4.

TABLE 4
Tooth-Whitening Composition having 5% H₂O₂

| | Component | |
|---|---|---|
| | A | B |
| | Peroxide Component | Alkaline Component |
| Ingredients | Weight % | Weight % |
| Deionized Water | 24 | 30.2 |
| Hydrogen peroxide | 10 | — |
| Potassium Nitrate | — | 10 |
| Polyox (PEG 2M) | 5 | — |
| Glycerin | 5 | 25 |
| Polyethylene glycol 600 | 10 | 3 |
| Xanthan | — | 0.7 |
| Na carboxymethyl cellulose | — | 0.5 |
| PLURONIC ® F-127 | 20 | 2 |
| Sodium saccharin | — | 0.2 |
| Titanium dioxide | — | 1 |
| Sodium hydroxide | — | 1 |
| (50% concentration) | | |
| TiO₂ | — | 1 |
| FD&C Green #3 | — | 0.025 |
| ZEODENT ® 115 (silica | — | 17.5 |
| abrasive) | | |
| ZEODENT ® 165 | — | 3 |
| (silica thickener) | | |
| Sodium bicarbonate | — | 5 |
| Calcium pyrophosphate | 25 | — |
| Sodium lauryl sulfate | — | 0.4 |
| Flavor | 0.5 | 0.5 |

Components A and B were prepared as described above in Example 1.

### EXAMPLE 5

This example illustrates the in vivo assessment of whitening efficacy of the composition of Example 4.

The composition of Example 4 was prepared by combining equal weight amounts of Components A and B to deliver 5% hydrogen peroxide. Whitening efficacy of the composition was also compared to that of comparative composition C2 containing 7.5% hydrogen peroxide in a two week human clinical study wherein one cell of 9 subjects used composition C2 thirty minutes twice daily according to the manufacturer's instruction. The other cell with 12 patients used the composition of Example 3 for fifteen minutes once daily. Tooth shade was evaluated after 0, 5, 7 and 14 days using a value-ordered Vita shade guide. The results are summarized in Table 5.

TABLE 5
In vivo Whitening Efficacy of Composition Containing 5% H₂O₂
Shade Guide Change

| | | | | | Average | | |
|---|---|---|---|---|---|---|---|
| | | Average | | | Shade Guide | | |
| | Average | Shade Guide | | | Change | | |
| | Shade at | (Day) | | | (Day) | | |
| Composition | Baseline | 5 | 7 | 14 | 5 | 7 | 14 |
| Example 5 | D3 | 7.67 | 6.15 | 4.88 | 3.58 | 4.92 | 6.19 |
| Comparative | D3 | 8.06 | 6.11 | 4.22 | 2.28 | 3.92 | 5.66 |
| Example C2 | | | | | | | |

The results recorded in Table 5 indicate that the overall whitening efficacy of the composition of Example 5 was directionally better than comparative composition C2 and statistically better at five days (p=0.025) evidencing faster whitening efficacy since both the application time and number of treatments using the composition of Example 5 was one quarter that of comparative composition C2 (fifteen minutes once daily versus thirty minutes twice daily). In addition, Example 5 contained 2.5% less hydrogen peroxide than comparative composition C2.

In addition to enhanced efficacy, the patients in the tooth whitening study using the composition of Example 5 as described above reported less gingival irritation and tooth sensitivity than patients using comparative composition C2. The patients involved in the study rated their tooth sensitivity and gingival irritation on a scale of zero (none) to 5, the higher the number, the greater the tooth sensitivity and gingival irritation experienced by the patient involved in the study. Patients rated their perception of tooth sensitivity and gingival irritation using this scale before using the product (baseline) and after using the product for seven days and fourteen days. The patient ratings of gum irritation and tooth sensitivity minus the baseline ratings are recorded in Table 6 below.

TABLE 6
Sensitivity and Irritation Study with Composition of Example 5

| | |
|---|---|
| Δ Gum | Δ Tooth |
| Irritation* | Sensitivity** |
| (Day) | (Day) |

| Composition | 7 | 14 | 7 | 14 |
|---|---|---|---|---|
| Example 5 | −0.08 | 0.00 | −0.62 | −0.46 |
| Comparative | +1.11 | +1.13 | 0.00 | +0.33 |
| Example C2 | | | | |

*Change in gum irritation from baseline
**Change in tooth sensitivity from baseline

Ratings for gum irritation and tooth sensitivity were lower for the composition of Example 5 compared to that of comparative composition C2 at seven and fourteen days. The levels of tooth sensitivity reported for the composition of Example 5 at seven and fourteen days were negative (lower than the reported baseline values), suggesting that the desensitizing agent present in the composition reduced tooth sensitivity over the treatment period.

The description of the invention is merely exemplary in nature and, thus, variations that do not depart from the gist of the invention are intended to be within the scope of the invention. Such variations are not to be regarded as a departure from the spirit and scope of the invention.

1. A dual component tooth-whitening system comprising:
a first component comprising a peroxide compound and a peroxide-compatible abrasive compound; and
a second component comprising an alkaline compound, wherein upon combining the first and second components the resultant tooth-whitening composition has a pH of about 9 or greater.

2. A system according to claim 1, wherein the first component has a pH of about 4 to about 7 and the second component has a pH of about 9 to about 13.

3. A system according to claim 1, wherein the peroxide compound is selected from the group consisting of hydrogen peroxide, an organic peroxide compound, a hydrogen peroxide generating compound, and combinations thereof.

4. A system according to claim 1 wherein the peroxide compound is present in the tooth-whitening composition at a hydrogen-peroxide-equivalent concentration of not greater than about 7.5% by weight.

5. A system according to claim 1 wherein the peroxide compound is present in the tooth-whitening composition at a hydrogen-peroxide-equivalent concentration of not greater than about 5% by weight.

6. A system according to claim 1, wherein the peroxide compound is present in the tooth-whitening composition at a hydrogen-peroxide-equivalent concentration of not greater than about 3.5% by weight.

7. A system according to claim 1, wherein the peroxide-compatible abrasive is selected from the group consisting of: calcium phosphate salts, dicalcium phosphate dihydrate, anhydrous dicalcium phosphate, calcium pyrophosphate, and mixtures thereof.

8. A system according to claim 1, wherein the peroxide-compatible abrasive is present in the tooth-whitening composition at a concentration of at least about 20% by weight.

9. A system according to claim 1, wherein the alkaline compound is selected from an alkali metal hydroxide and an alkali metal carbonate salt.

10. A system according to claim 1, wherein the second component further comprises an abrasive compound.

11. A system according to claim 1, wherein the second component further comprises a color indicator selected from the group consisting of FD&C Red No. 3, FD&C Yellow No. 5, FD&C Yellow No. 6, FD&C Green No. 3, FD&C Blue No. 1 and combinations thereof.

12. A system according to claim 1, wherein either or both of the first or second components further comprise a tooth-desensitizing compound.

13. A system according to claim 12, wherein the tooth-desensitizing compound is selected from a potassium salt of a weak acid and eugenol.

14. A system according to claim 1, wherein the second component comprises one or more stabilizing compounds compatible with the alkaline compound.

15. A system according to claim 14, wherein the one or more stabilizing compounds are selected from the group consisting of: agar, furcellan algin, chitin, chitosan, carrageenan, xanthan gum, gum arabic, dextran, Irish moss, gum tragacanth, starch, hydroxyethyl cellulose, hydroxypropyl cellulose), polyvinylpyrrolidone, hydroxyethyl propyl cellulose, hydroxybutyl methyl cellulose, hydroxypropyl methyl cellulose, hydroxyethyl cellulose, sodium carboxymethyl cellulose, colloidal silica, and mixtures thereof.

16. A system according to claim 14, wherein the one or more stabilizing compounds are selected from the group consisting of: xanthan gum, carboxymethyl cellulose, and mixtures thereof.

17. A system according to claim 14, wherein the one or more stabilizing compounds comprise a first stabilizing compound and a second stabilizing compound provided in a ratio of about 2:1 to about 1:4, respectively.

18. A system according to claim 1, wherein the tooth-whitening system is substantially free of a chelating agent.

19. A system according to claim 1, wherein the tooth-whitening system further comprises an oral care active agent selected from the group consisting of: an anticaries agent, a tartar control agent, a periodontal active, a breath freshening agent, a malodor control agent, a nutrient, an anti-inflammatory agent, a salivary stimulant, an anti-adhesion agent, a plaque dispersing agent, and mixtures thereof.

20. A tooth-whitening composition comprising a peroxide compound at a hydrogen-peroxide-equivalent concentration of not greater than about 7.5% by weight and a peroxide-compatible abrasive compound, wherein the composition has a pH of about 9 or greater.

21. A composition according to claim 20, wherein the peroxide compound is at a hydrogen-peroxide-equivalent concentration of not more than about 5.0% by weight.

22. A composition according to claim 20, wherein the peroxide compound is at a hydrogen-peroxide-equivalent concentration of not more than about 3.5% by weight.

23. A composition according to claims 20, wherein the composition produces a tooth-whitening effect equal to or greater than that of a comparative tooth-whitening composition having a hydrogen peroxide concentration of 7% by weight or greater and a pH of 7 or less.

24. A composition according to claim 20, wherein a concentration of the abrasive compound is at least about 20% by weight.

25. A composition according to claim 20, further comprising a color indicator.

26. A composition according to claim 20, further comprising a compound selected from a potassium salt of a weak acid and eugenol.

27. A method for decreasing whitening time, increasing whitening effectiveness or decreasing oral irritation in a tooth-whitening process, the method comprising contacting the tooth for an effective tooth-whitening period with a tooth-whitening composition that comprises a peroxide compound at a hydrogen-peroxide-equivalent concentration of not greater than about 7.5% by weight and a peroxide-compatible abrasive compound, the composition having a pH of at least about 9.

28. A method according to claim 27, wherein the peroxide compound is at a hydrogen-peroxide-equivalent concentration of not greater than about 5.0% by weight.

29. A method according to claim 27, wherein the peroxide compound is at a hydrogen-peroxide-equivalent concentration of not greater than about 3.5% by weight.

30. A method according to claim 27, wherein the composition is prepared from a dual component system comprising a first component comprising a peroxide compound and a peroxide-compatible abrasive compound and a second component comprising an alkaline compound.

31. A method according to claim 30, wherein the composition is prepared by combining the first component and the second component together not more than fifteen minutes prior contacting the tooth with the composition.

32. A method according to claim 30, wherein the second component further comprises a color indicator.

33. A method according to claim 27, wherein the effective tooth-whitening period is less than that required for a comparative composition comprising hydrogen peroxide at a concentration of 7% by weight or greater and a pH of about 7 or less.

34. A method according to claim 27, wherein the tooth-whitening composition has a concentration of the abrasive compound in the composition of at least about 20% by weight.

35. A method according to claim 27, wherein the tooth-whitening composition further comprises a tooth-desensitizing compound.

36. A dual component tooth-whitening system comprising:
a first component comprising a peroxide compound and a peroxide-compatible abrasive compound; and
a second component comprising an alkaline compound and one or more stabilizing compounds compatible with the alkaline compound, wherein upon combining the first and second components the resultant tooth-whitening composition has a pH of about 9 or greater.

37. A system according to claim 36, wherein the one or more stabilizing compounds is selected from the group consisting of: agar, furcellan algin, chitin, chitosan, carrageenan, xanthan gum, gum arabic, dextran, Irish moss, gum tragacanth, starch, hydroxyethyl cellulose, hydroxypropyl cellulose), polyvinylpyrrolidone, hydroxyethyl propyl cellulose, hydroxybutyl methyl cellulose, hydroxypropyl methyl cellulose, hydroxyethyl cellulose, sodium carboxymethyl cellulose, colloidal silica, and mixtures thereof.

38. A system according to claim 36, wherein the one or more stabilizing compounds is selected from the group consisting of: xanthan gum, carboxymethyl cellulose, and mixtures thereof.

39. A system according to claim 36, wherein the one or more stabilizing compounds comprise a first stabilizing compound and a second stabilizing compound in a ratio of about 2:1 to about 1:4, respectively.
